(19) United States
(12) Patent Application Publication
(10) Pub. No.: US 2005/0053651 A1
(43) Pub. Date: Mar. 10, 2005

(54) Stable pharmaceutical formulation

(76) Inventors: Armin Knapp, Gottenheim (DE); Sven Schreder, Ulm (DE)

Correspondence Address: WARNER-LAMBERT COMPANY, 2800 PLYMOUTH RD, ANN ARBOR, MI, 48105, US

(21) Appl. No.: 10/891,779

(22) Filed: Jul. 15, 2004

(30) Foreign Application Priority Data
Jul. 15, 2003 (GB) 03016056.8

(51) Int. Cl. A61K 9/64

(52) U.S. Cl. 424/456

(57) ABSTRACT

The invention concerns a pharmaceutical composition in gelatine capsule form, comprising (a) an active core composition comprising at least one pharmaceutically active compound and optionally conventional pharmaceutical adjuvants, (b) at least one quenching protective layer, positioned between said active core composition and the inner surface of the gelatine capsule shell, which comprises at least one quenching agent, the quenching agent being able to chemically bind volatile impurities initiating the cross-linking of gelatine by a substantially irreversible chemical reaction after said impurities have been released by either adjuvants and/or active compound(s) of said active core composition and (c) a gelatine capsule shell wrapping said active core composition including any adjuvant material and any quenching layer added to the active core composition. Such a composition has improved stability on storage.

### STABLE PHARMACEUTICAL FORMULATION

This application is a United States utility application, which claims the benefit of priority to United Kingdom patent application Serial No. 03016056.8 filed Jul. 15, 2003.

This application concerns a controlled release pharmaceutical formulation in the form of a gelatine capsule which shows improved stability. This application further concerns a process for preparing such a composition and a method for improving the storage stability of a gelatine encapsulated controlled release composition. In a preferred aspect, the application relates to a controlled release pharmaceutical formulation in the form of a gelatine capsule comprising eletriptan, or a salt thereof, which shows improved stability, and the use of such a formulation in the treatment of migraine and in the prevention of migraine recurrence.

It is desirable, in many cases, to provide a controlled release of a drug. Such a controlled release is often achieved by coating a multiparticulate composition comprising the active compound (e.g. beads, pellets, granulates, mini-tablets, mini- or microcapsules or caplets) with a commercially available aqueous dispersion such as an ammonio methacrylate copolymer, particularly a Eudragit® copolymer manufactured by Röhm Pharma GmbH. Such coated particulates are then usually dispensed into commercially available capsules, in most cases gelatine-capsules, for ease of administration.

An example of such a gelatine-encapsulated controlled release formulation is described in WO-A-02/09675 in relation to the antimigraine drug eletriptan. This patent application describes a a pharmaceutical composition in particulate form, suitable for oral administration, including a core containing eletriptan or a pharmaceutically acceptable salt thereof, the core being coated with a water-insoluble, permeable coating including one or more acrylic copolymer(s) containing [symbol] thylammoniumethylmethacrylate groups, said composition being capable of achieving a sigmoidal pattern of controlled drug release. The acrylic copolymer(s) is/are preferably selected from Eudragit RL® and Eudragit RS® and the particulate compositions described are preferably filled into hard gelatine capsules before administration.

In order to obtain a fast-acting initial dose, such a controlled release formulation comprising a drug core coated with ammonio methacrylate copolymers, may be combined with a further portion of drug in immediate release form in the same capsule (see Example 7 of WO-A-02/09675). Alternatively, multiparticulate compositions with different controlled release profiles may be combined in one capsule. Such formulations are known as dual release formulations.

Such gelatine capsule preparations, once prepared, are usually stored for a period of time before use. The storage period may extend to months or even years and the conditions under which the preparations are stored may vary in terms of temperature and humidity. Clearly, the pattern of controlled release that an encapsulated preparation demonstrates when administered to a patient should not change during the storage period or the wrong dose of drug may be delivered to the patient at the wrong time.

Surprisingly, it has been found that gelatine capsule preparations manufactured in accordance with the teaching of WO-A-02/09675, containing a dual release formulation of eletriptan, are not stable on storage, particularly under stressing conditions (i.e. relatively high temperature and/or humidity). It appears that the dissolution rate of the capsule shell changes over a period of time leading to unexpected changes in the release profile of the immediate and/or controlled release particles contained in the capsule. Such changes in the capsule shell appear to be a general feature of gelatine capsules filled with multiparticulate compositions that are coated with aqueous dispersions of ammonio methacrylate copolymers such as Eudragit® RL 30 D (type A) or Eudragit®) RS 30 D (type B) or with compositions that are produced from a solid ammonio methacrylate copolymer such as Eudragit® RL PO. In a dual release formulation the changes in programmed release occur unpredictably as either a delay in the initial release of drug from immediate release particles and/or, by interaction of formulation ingredients (e.g. by interaction of soluble, rapid-release anionic components with the ammonio methacrylate copolymers), as accelerated release of drug from controlled-release particles.

Such unpredictable changes in drug release profile from a gelatine-encapsulated formulation comprising ammonio methacrylate copolymers is quite unprecedented and the consequent unacceptable variability in drug release profile has been hitherto unrecognised. There therefore exists a need to provide means to stabilise such a formulation, allowing long term storage at varying temperatures and levels of humidity without any change in the dissolution rate of the capsule shell and any consequent changes in the release profile of immediate or controlled-release particles contained therein.

We have now unexpectedly found that such means can be provided by an incorporating a quenching agent in the formulation.

The invention therefore provides a pharmaceutical composition in gelatine capsule form, comprising (a) an active core composition comprising at least one pharmaceutically active compound and optionally conventional pharmaceutical adjuvants, (b) at least one quenching protective layer, positioned between said active core composition and the inner surface of the gelatine capsule shell, which comprises at least one quenching agent, the quenching agent being able to chemically bind volatile impurities initiating the cross-linking of gelatine by a substantially irreversible chemical reaction after said impurities have been released by either adjuvants and/or active compound(s) of said active core composition and (c) a gelatine capsule shell wrapping said active core composition including any adjuvant material and any quenching layer added to the active core composition.

In a preferred embodiment the active core composition comprises an ammonio methacrylate copolymer such as Eudragit® RL 30 D (type A) or Eudragit® RS 30 D (type B), most preferably as a controlled release coating on the external surface of the active core composition.

Thus, the invention also provides a multiparticulate composition, suitable for administration in a gelatine capsule, comprising a drug-containing core, said core being coated with a controlled release coating comprising an ammonio methacrylate copolymer, wherein the composition comprises a quenching agent in the controlled release coating or in an outer quenching protective layer.

The invention further provides a method for improving the stability of a pharmaceutical gelatine-capsule preparation by preventing or reducing the cross-linking of the gelatine material in the capsule shell initiated by volatile impurities that are released by (a) preparing a quenching protective layer comprising at least one quenching-agent which is able to chemically bind volatile impurities initiating the cross-linking of gelatine, by a substantially irreversible chemical reaction and (b) positioning said quenching layer in the space between the pharmaceutical core compositions or its components releasing volatile impurities and the inner surface of the gelatine-capsule shell in a way that said volatile impurities are forced to contact and penetrate the surface of said quenching protective layer before said volatile impurities are able to contact the inner surface of the gelatine capsule shell.

A quenching agent is a compound that can remove volatile impurities, particularly aldehydes, by binding them irreversibly and preventing their diffusion into and reaction with the gelatine present in the capsule shell. Such a quenching agent may be an amine, protein, amino-acid, oligo- or polypeptide, especially gelatine and/or an amino-acid. Particularly preferred quenching agents are cysteine, lysine, aspartic acid, asparagine, gelatine and glycine. Most preferred are glycine and aspartic acid.

The quenching agent can be incorporated into a quenching layer which comprises, in addition to the quenching agent, a water soluble film forming material, particularly a hydrophilic polymer such as hydroxypropyl-methylcellulose or hydroxypropyl cellulose. The quenching agent preferably comprises 1% to 60% by weight of such a quenching layer. Hydroxypropyl-methylcellulose is highly soluble in water and body liquids and used as a quenching layer has a minimal influence on the desired profile of drug release.

The quenching agent may be applied in several ways to form a barrier that neutralises volatile impurities. A first example is a gelatine capsule preparation, wherein a quenching protective layer, comprising the quenching agent, is positioned next to or coated onto the inner surface of the gelatine capsule shell. A further possibility is a gelatine capsule preparation, wherein a quenching protective layer is positioned next to or coated onto the outer surface of the active core composition. A further method is to integrate the quenching agent into the controlled release layer.

Immediate release and controlled release pellets may be prepared according to the procedures described in WO-A-02/09675 and a quenching protective layer may then be applied, preferably at a level of from 1 to 10% by weight, preferably from 1 to 3% by weight. Where gelatine is employed as a quenching agent, the highest possible amount should be applied with the proviso that the stability and solubility of the quenching layer and also the desired retarding effect is not unacceptably deteriorated. This amount may account from 1 to 25%, preferably 1 to 15% by weight, of the final particulate composition.

The use of a quenching layer in accordance with the invention has further advantages in certain circumstances. For instance, when controlled release beads were manufactured in accordance with the procedures of WO-A-02/09675 and the beads obtained were filled into hard gelatine capsule shells using automatic capsule-filling machines with pellet-filling stations, severe scratching of the machine parts occurred and the scratching noise was even heard while the machine was running. The process had to be stopped and machine parts of the capsule-filling stations had to be replaced because of deep scratching traces on the surface. Surprisingly it was found, that application of a quenching protective layer with coating levels of from 10 to 25% by weight, preferably from 10 to 15 % by weight, improved the capsule-filling properties and allowed the filling of commercial batch sizes without any technical difficulties and without the damages described above.

One application of the invention is the use of a quenching agent in “caplets” according to EP-A-0891180. Such caplets may be included into gelatine-capsule shells. They comprise a coating composition comprising cellulose-acetate-phthalate (cellacephate), polyvinyl acetate-phthalate, methacrylic acid polymers, hypromellose-phthalate, hydroxyalkyl-methylcellulose-phthalates or mixtures thereof.

The quenching protective layer may be prepared as an additional water-soluble layer containing e.g. an amino acid. In the case of caplets it is also possible to add e.g. gelatine to the above coating before placing the caplet in the capsule shell. The film-forming agent in the case of caplets is used in an amount of about 20% of weight. Glidants and or adhesives for fixing the caplet in the shell may be added. If gelatine and/or an amino-acid is added to the film forming material as the quenching barrier-substances, the amount may be 1 to 50 weight % of the film forming material.

It is believed that the significant changes in the release characteristics of a gelatine encapsulated controlled release formulation described above are brought about as a result of an interaction between the gelatine capsule and volatile impurities in the ammonio methacrylate copolymers which leads to cross-linking of the gelatine. Such impurities are thought to be low molecular weight aldehydes. This cross-linking process, which increases as a function of time and seems to be exacerbated by higher humidity and/or higher temperature, results in a markedly slower disintegration of the gelatine capsule when administered into an aqueous environment and a corresponding change in the release of active compounds contained therein.

This cross-linking effect is responsible for release delays of up to 12 minutes, especially if the capsule contains immediate release components, while the standard disintegration time of a freshly prepared capsule is only about 3 minutes. Strongly cross-linked gelatine capsules may be even totally resistant to gastric juice. In less cross-linked preparations the release time of the pharmacologically active particulate components is still delayed by 5 to 10 minutes. In the case of pharmaceuticals such as analgesics or antimigraine agents, where a rapid onset of action is required, such a delay in disintegration time is clearly unacceptable.

In the case of a dual release formulation, the longer disintegration time of the gelatine capsule seems to lead to unwanted, deleterious interactions between the particles programmed to have different release characteristics. This is because the capsule shell may partially leak relatively early e.g. as early as 3 minutes after being in contact with gastric juice, while maintaining its main structure and not allowing the release of particulate components for another 10 minutes before the complete break-up of the capsule-shell. In the “time-gap” of about 7 minutes, fast releasing components dissolve almost immediately inside the capsule-shell and form a highly concentrated solution of soluble ingredients which has sufficient time to interact with still intact slower releasing components. Osmotic influences and different ion-size, ion-charge and ion-hydration of e.g. phosphate, citrate, methansulfonate, bromide or sulphate anions in comparison to e.g. chloride ions may cause severe damage to the controlled release layer of the slower releasing components, and these undesired interactions change the release characteristics of the slower releasing components, often to an unacceptable degree.

In the case of the dual release formulation described in WO-A-02/09675, for instance, chloride anions from the ammonio methacrylate copolymers may exchange with sulphate anions from the immediate release portion of eletriptan hemisulphate, altering the properties of the controlled release coating and thus causing a significantly accelerated release of eletriptan from the controlled release particles. Such an interaction between chloride and sulphate anions takes place only if the capsule-shell is crosslinked and therefore hinders diffusion of sulphate anions from the immediate release portion out of the capsule during contact with gastric or intestinal juice.

It is believed that the quenching agent provided by the present invention isolates and removes volatile impurities, such as low molecular weight aldehydes, emanating from ammonio methacrylate copolymers that would otherwise trigger or initiate the cross-linking of the gelatine capsule shell.

In order to further reduce cross-linking of the gelatine-shell, the level of volatile impurities in the formulation, especially in the ammonio methacrylate layer, should be kept to a minimum. As discussed above, the most important volatile impurities are thought to be aldehydes, especially lower aliphatic aldehydes with up to 6 carbon atoms, particularly formaldehyde and acetaldehyde. The aldehyde content of the particles contained within the gelatine capsule should therefore preferably be less than 10 ppm, more preferably less than 5 ppm and most preferably less than 2 ppm.

The level of volatile aldehyde impurities may be reduced by vacuum treatment of the ammonio methacrylate-containing particles (e.g. at less than 100 mbar, preferably 1-20 mbar), especially at raised temperatures (e.g. 25-50° C., preferably 35-45° C.). Alternatively, the particles may be exposed to a stream of clean and dry air or nitrogen at raised temperatures (30-50° C., preferably 35-45° C.) in a fluidized bed unit. The air or nitrogen should have a relative humidity of less than 20%.

The ingredients of the total capsule contents as well as the capsule forming gelatine must be as clean as possible before the capsule and/or its contents are finally prepared and composed.

The term gelatine capsule or gelatine capsule preparation according to this invention refers to a capsule material with a gelatine content of about 20 to 100 weight %. Capsule material containing less than 100% by weight gelatine comprise usual adjuvants known to a person skilled in the pharmaceutical capsule manufacturing art, such as water-soluble polymers.

In a preferred aspect of the invention, the ammonio methacrylate copolymer is a type A or a type B ammonio-methacrylate copolymer (e.g. the products marketed under the trade-mark Eudragit RL and Eudragit RS) or a mixture of the two. These copolymers contain chloride counter-ions.

If desired, a particulate composition according to the invention may be coated with a so-called top-coat (see, for instance, WO-A-02/09675) which conventionally has a hydrophilic character. This is a usual measure in the field of gelatine capsule manufacture for improving the mechanical properties of the particulate composition with regard to its further processing (mixing, transport, filling into capsule-shells).

The ammonio methacrylate coating applied to the drug-containing particles may include one or more additional substances, such as a softening agent (e.g. an acetylated monoglyceride, triethyl citrate, acetyl-triethyl citrate, tributyl citrate, acetyltributyl citrate, another citrate ester, dibutyl phthalate, diethyl phthalate, another phthalate ester, diethyl sebacate, dibutyl sebacate, diethyl fumarate, diethyl succinate, a polyethylene glycol, glycerol, sesame oil, a lanolin alcohol or triacetin), an anti-tacking agent (e.g. talc, calcium stearate, colloidal silicon dioxide, glycerin, magnesium-stearate, mineral oil, a polyethylene glycol, zinc- stearate, aluminium-stearate or glycerol-monostearate), a wetting agent (e.g. sodium-lauryl-sulphate, stearyl alcohol, acacia, benzalkonium chloride, cetomacrogol emulsifying wax, cetostearyl alcohol, cetyl alcohol, cholesterol, diethanolamine, sodium stearate, glycerol monostearate, hydroxypropyl-cellulose, a lanolin alcohol, triethanolamine, lecithin, poloxamer, a polyoxyethylene alkyl ether, a sorbitan ester, a stearyl alcohol or simethicone or a water insoluble polymer (e.g. ethyl-cellulose, cellulose-acetate or a poly-methacrylate copolymer). A preferred softening agent is triethyl-citrate, a preferred anti-tacking agent is talc and a preferred wetting agent is sodium-lauryl-sulphate.

A preferred drug for use in the invention is eletriptan, or a pharmaceutically acceptable salt or solvate thereof. Particularly preferred are eletriptan hydrobromide and eletriptan hemisulphate. Most preferred is eletriptan hemisulphate. Eletriptan and its salts and solvates are useful in the treatment of migraine and the prevention of migraine recurrence.

### BRIEF DESCRIPTION OF THE FIGURES

Other advantages of the present invention will be readily appreciated, as the same becomes better understood by reference to the following description when considered in connection with the accompanying Figures wherein:

FIG. 1 is a graph showing the release profile of eletriptan-hemisulfate dual release 40/40 capsules;

FIG. 2 is a graph showing the release profile of eletriptan-hemisulfate dual release 40/40 capsules;

FIG. 3 is a graph showing the release profile of eletriptan-hemisulfate dual release 40/40 capsules;

FIG. 4 is a graph showing the release profile of eletriptan-hemisulfate dual release 40/40 capsules;

FIG. 5 is a graph showing the release profile of eletriptan-hemisulfate dual release 40/40 capsules;

FIG. 6 is a graph showing the release profile of eletriptan-hemisulfate dual release 40/40 beads;

FIG. 7 is a graph showing the release profile of eletriptan-hemisulfate dual release 40/40 capsules;

FIG. 8 is a graph showing the release profile of eletriptan-hemisulfate dual release 40/40 capsules;

FIG. 9 is a graph showing the release profile of eletriptan-hemisulfate dual release 40/40 capsules;

FIG. 10 is a graph showing the release profile of eletriptan-hemisulfate dual release 40/40 capsules;

FIG. 11 is a graph showing the release profile of eletriptan-hemisulfate dual release 40/40 capsules;

FIG. 12 is a graph showing the release profile of eletriptan-hemisulfate dual release 40/40 capsules;

FIG. 13 is a graph showing the release profile of eletriptan-hemisulfate dual release 40/40 capsules;

FIG. 14 is a graph showing the release profile of eletriptan-hemisulfate dual release 40/40 capsules;

FIG. 15 is a graph showing the release profile of eletriptan-hemisulfate dual release 40/40 capsules;

FIG. 16 is a graph showing the release profile of eletriptan-hemisulfate dual release 40/40 capsules;

FIG. 17 is a graph showing the release profile of eletriptan-hemisulfate dual release 40/40 capsules; and

FIG. 18 is a graph showing the release profile of eletriptan-hemisulfate dual release 40/40 capsules.

A non-limiting example of a typical capsule preparation according to the present invention is as follows:

1. A core composition
The inner core composition contains an active compound in the form of crystals, granulates, pellets, spheric extrudates, spray dried products, minitablets, microcapsules or minicapsules in an amount of from 10 to 90% by weight.

2. A barrier coating
This optional layer contains a hydrophilic polymer such as hydroxypropyl methylcellulose in an amount of from 1 to 20% by weight.

3. A controlled release layer
This layer controls the release of the active compound or compounds and preferably contains
a. a water-insoluble polymer such as an ammonio methacrylate-copolymer, type A and/ or B (or a mixture thereof) in an amount of from 10 to 50% by weight,
b. a softening agent such as triethyl-citrate in an amount of from 1 to 15% by weight,
c. an anti-tacking agent such as talc in an amount of from 3 to 50% by weight.

4. A quenching protective layer
This layer contains, for example, gelatine, glycine, asparagine or cysteine in an amount of from 0.2 to 2% by weight.

5. A barrier coating
This optional layer, which is the same as layer (2) described above, is present in an amount of from 10 to 20% by weight.

6. The gelatine containing capsule-shell
The above example is only a typical formulation according to the invention. Layer 4 as well as the outer barrier coating may be put in different locations. Instead of a separate quenching protective layer, the quenching agent may be incorporated in the outer barrier coating or the controlled release layer 3 and thus form a bifunctional or multifunctional layer. The preparation may also contain additional protective layers of both kind and any practical number of top-coats or other useful layers known in the art.

The present invention provides a storage stable gelatine-capsule preparation which in a customary packaging may be stored over a long time, even under stressing conditions such as, for example, relatively high temperatures of above 30° C. and a high humidity of more than 50%. The invention is especially useful for capsule preparations for use in tropical regions.

The capsules of the invention maintain a release profile that is substantially identical to the initial product before storage.

The following examples only illustrate the present invention and may under no circumstances be seen as a limitation of the inventive concept.

### EXAMPLE 1

### Changes to the Release Profile of a Prior Art Dual Release Formulation on Storage

A dual release formulation comprising eletriptan hemisulphate immediate release beads and eletriptan hemisulphate controlled release beads in a gelatine capsule was prepared using the procedures of WO-A-02/09675.

Eletriptan hemisulphate starter cores were coated first with the Eudragit polymers to create the controlled release coating and then with hydroxypropyl methylcellulose (Opadry) as a topcoat. The weight of the finished controlled release beads was 210.48 mg.

Eletriptan hemisulphate controlled release beads

| Component | Description | Amount |
|---|---|---|
| Eletriptan hemisulphate starter | Core composition | 115.51 mg |
| cores (corresponding to 40.00 | (including a barrier coat) | |
| mg eletriptan base) | | |
| Ammonio metacrylate copoly- | Controlled release | 40.62 mg |
| mer Type B (Eudragit ® RS) | coating | |
| Ammonio methacrylate copoly- | | 2.14 mg |
| mer Type A (Eudragit ® RL) | | |
| Triethylcitrate NF | | 8.54 mg |
| Talc | | 21.37 mg |
| Opadry ® Yellow YS-1- | Top coat | 22.30 mg |
| 12570-A | | |

The controlled release beads were cured in a tray oven at 40° C. for 24 hours and then filled together with immediate release beads into hard gelatine capsules (Size 1) using a Bosch GKF 400 capsule filling machine. The capsule fill weight was 325.99 mg.

Eletriptan hemisulphate dual release capsule

| Component | Description | Amount |
|---|---|---|
| Eletriptan hemisulphate immediate | Core composition | 115.51 mg |
| release beads (corresponding to | including top coat | |
| 40.00 mg eletriptan base) | | |
| Eletriptan hemisulphate controlled | Core Composition | 210.48 mg |
| release beads (corresponding to | including barrier coat, | |
| 40.00 mg eletriptan base) | controlled release layer | |
| | and top coat | |
| Hard gelatine capsule shell size 1, | Capsule shell | 76 mg |
| two-tone grey | | |

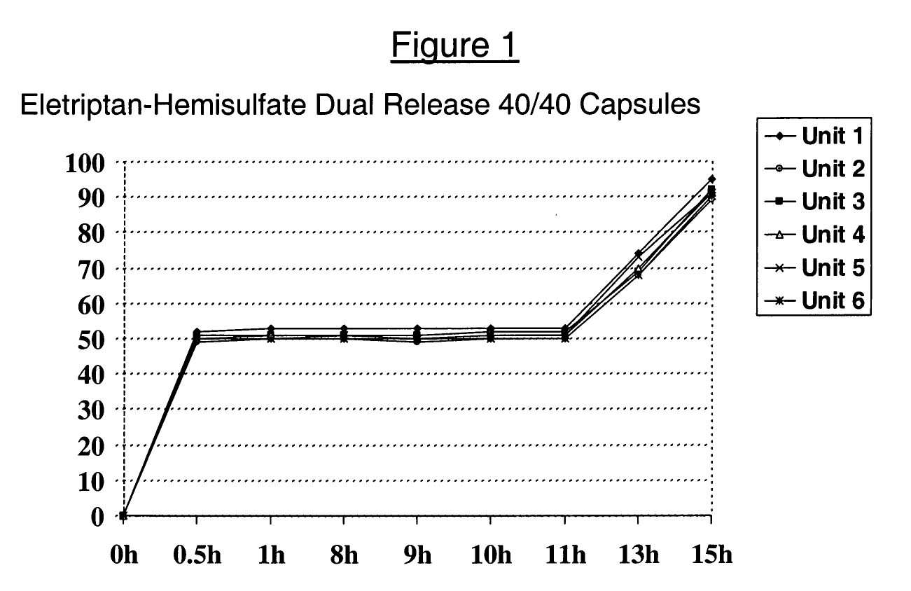

The release profile as shown in FIG. 1 was obtained using the dissolution test described in Example 6 of WO-A-02/09675.

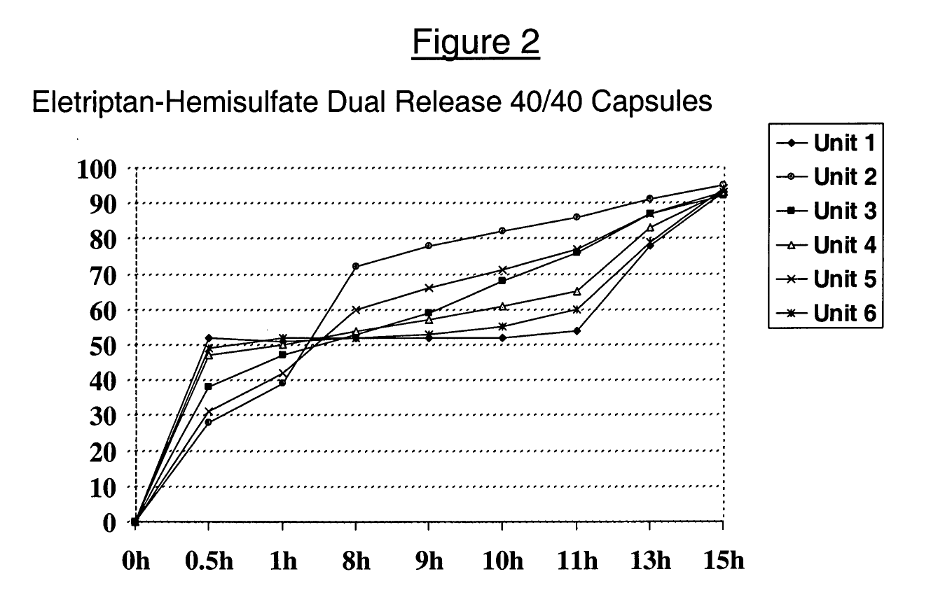

After storage for 4 weeks at 40° C./75% relative humidity in HDPE bottles the dissolution test was repeated and the following very different results were obtained and are shown in FIG. 2.

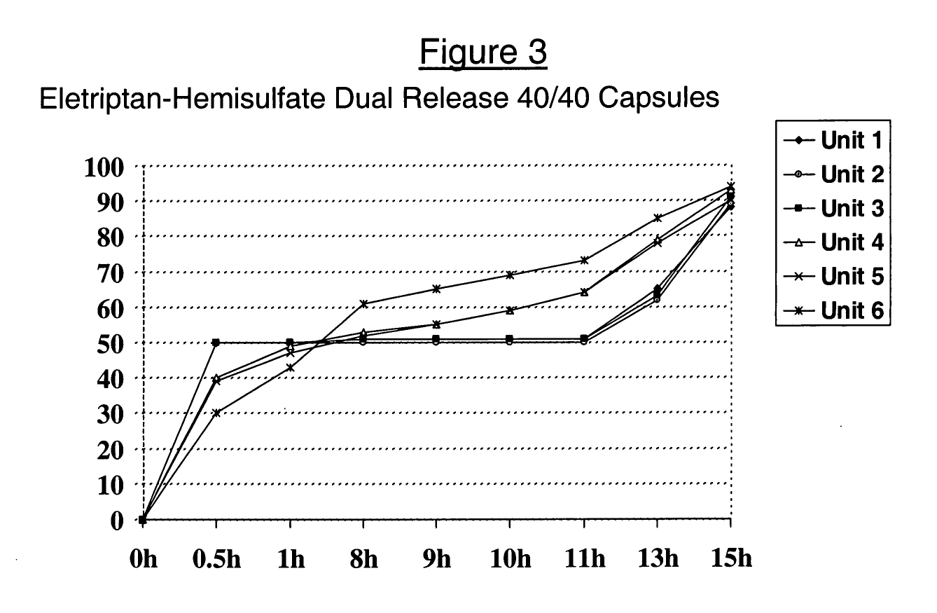

Storage for 6 weeks at 30° C./60% relative humidity in HDPE bottles resulted also in very significant changes. The results are shown in FIG. 3.

Thus, the dissolution profile of a gelatine-encapsulated dual release formulation of eletriptan, made in accordance with the procedures described in WO-A-02/09675, shows considerable variability when the formulation is stored in HDPE bottles under at least 2 different storage conditions (40° C./75% relative humidity and 30° C./60% relative humidity).

### EXAMPLE 2

### Changes to the Release Profile of a Prior Art Dual Release Formulation on Storage

A further dual release formulation comprising eletriptan hemisulphate immediate release beads and eletriptan hemisulphate controlled release beads in a gelatine capsule was prepared using the procedures of WO-A-02/09675.

Eletriptan hemisulphate starter cores were coated first with the Eudragit® polymers to create the controlled release coating and then with hydroxypropyl methylcellulose (Opadry) as a topcoat. The weight of the finished beads was 175.40 mg.

Eletriptan hemisulphate controlled release beads

| Component | Description | Amount |
|---|---|---|
| Eletriptan hemisulphate starter | Core composition including | 105.54 mg |
| cores (corresponding to 40.00 | barrier coat | |
| mg eletriptan base) | | |
| Ammonio methacrylate | Controlled release layer | 36.66 mg |
| copolymer type B (Eudragit ® | | |
| RS) | | |
| Ammonio methacrylate | | 1.93 mg |
| copolymer type A (Eudragit ® | | |
| RL) | | |
| Triethylcitrate NF | | 7.71 mg |
| Talc | | 19.29 mg |
| Opadry ® Blue OY-LS-20925 | Top coat | 4.27 mg |

The controlled release beads were cured in a tray oven at 40° C. for 24 hours and then filled together with immediate release beads into hard 15 gelatine capsules (size 1) using a Bosch GKF 400 capsule filling machine. The capsule fill weight was 280.94 mg.

Eletriptan hemisulphate dual release capsule

| Component | Description | Amount |
|---|---|---|
| Eletriptan hemisulphate | Core composition including | 105.54 mg |
| immediate release beads | top coat | |
| (corresponding to 40.00 mg | | |
| eletriptan base) | | |
| Eletriptan hemisulphate | Core composition including | 175.40 mg |
| controlled relase beads | barrier coat, controlled | |
| (corresponding to 40.00 mg | release layer and top coat | |
| eletriptan base) | | |
| Hard gelatin capsule shell | Capsule shell | 76 mg |
| (Size 1), two-tone grey | | |

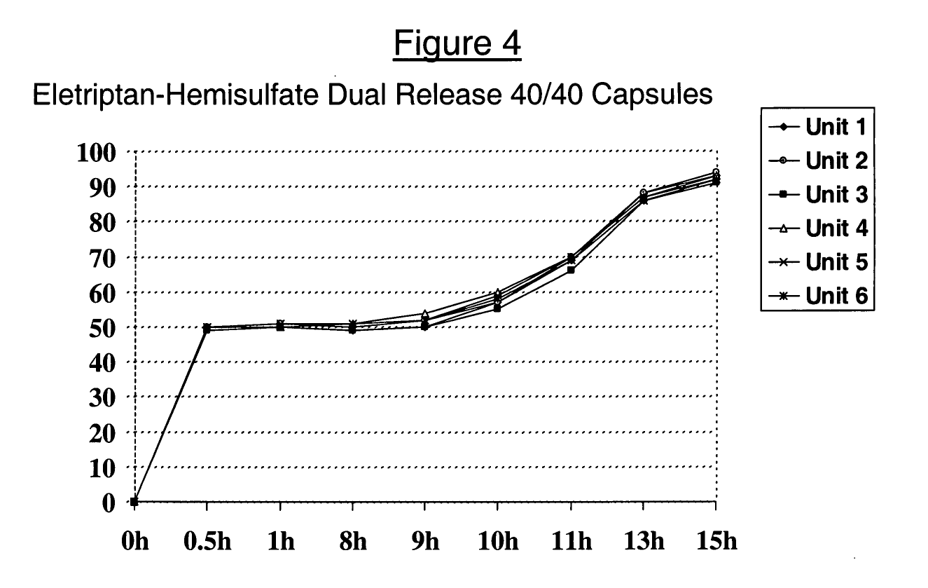

The release profile as shown in FIG. 4 was obtained using the dissolution test described in Example 6 of WO-A-02/09675.

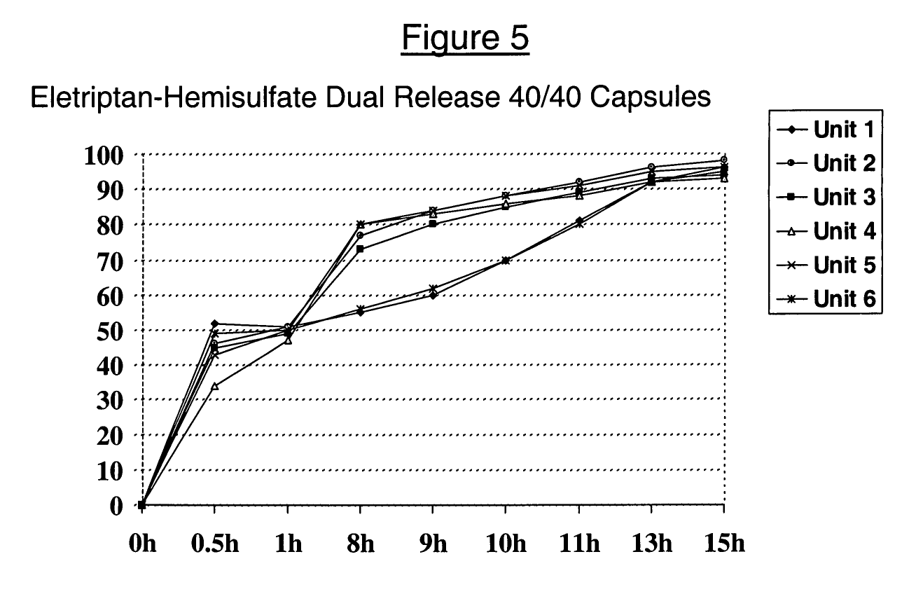

After storage for 3 months at 25° C./60% relative humidity in aluminium/aluminium foil/foil blisters the dissolution test was repeated and the following very different results (see FIG. 5) were obtained.

Similarly, 3-month storage at 40° C./30% relative humidity in PVC/Aclar white opaque blisters, 3-month storage at 25° C./60% relative humidity in HDPE bottles, 6-week storage at 40° C./30% relative humidity in HDPE bottle and 6-week storage at 30° C./60% relative humidity in HDPE bottles all resulted in significant changes to the release profile.

These experiments show that the dissolution profile of a gelatine-encapsulated dual release formulation made in accordance with the procedures described in WO-A-02/09675 shows considerable variability when stored under different storage conditions (i.e. 25° C./60% relative humidity, 30° C./60% relative humidity, 40° C./30% relative humidity and 40° C./75% relative humidity) and in different packaging materials (i.e. aluminium/aluminium foil/foil blisters, PVC/Aclar white opaque blisters and HDPE bottles).

### EXAMPLE 3

### Experiment to Determine Whether Changes in Stability Relate to Changes in the Gelatine Capsule or the Drug-Containing Beads

Dual release gelatine capsules made according to Example 2 were tested for dissolution, as described in Example 2, and found to give the expected release profile. They were then stored at 40° C./75% relative humidity in PVC/Aclar White Opaque blisters and retested. -The expected significant changes in dissolution profile were observed.

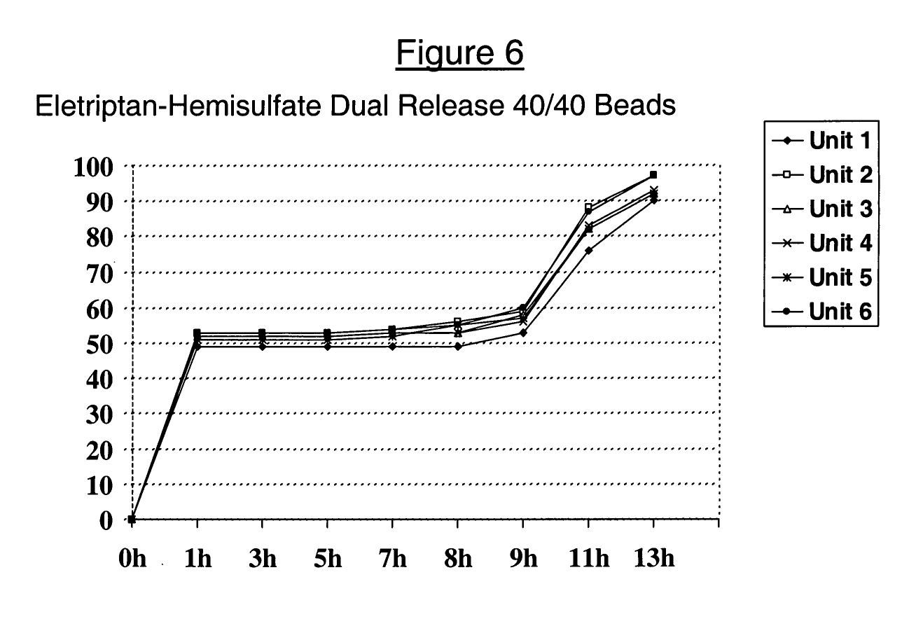

Capsules stored for the same time under the same conditions were opened and the beads were removed from the capsule shells and tested for dissolution. The following release profile as shown in FIG. 6 was observed.

Without capsule shells, bead dissolution was substantially unaffected after storage, showing that changes in dissolution profile are linked to changes in the capsule shell and not the beads themselves.

### EXAMPLE 4

### Changes in the Release Profile of Gelatine Capsules Containing Drugs Other than Eletriptan or No Drug at All

Minipress® retard 1 mg capsules (Pfizer) containing prazosin hydrochloride, medium-chain triglycerides, lactose monohydrate, talc, maize starch, saccharose, ammonio methacrylate copolymer, indigo carmine (E 132), erythrosin (E127), iron oxides (E 172), and titanium dioxide (E 171) were stored for 6 weeks at 40° C./75% relative humidity in HDPE bottles. The capsule shells were opened, emptied and filled with eletriptan immediate release beads (corresponding to 40 mg eletriptan) and eletriptan controlled release beads (corresponding to 40 mg eletriptan). A non-uniform release pattern of drug release was observed.

In a separate experiment, glass beads were coated with ammonio methacrylate copolymers type A and B and talc and filled into hard gelatine capsule shells (size 1). After 4-week storage of this placebo formulation (without any active ingredient) at 40° C./75% relative humidity in induction-sealed HDPE bottles, the capsule shells were opened, emptied and filled with eletriptan immediate release beads (corresponding to 40 mg eletriptan) and eletriptan controlled release beads (corresponding to 40 mg eletriptan). The capsule shells were closed again and subjected to a dissolution test. A non-uniform pattern of drug release was observed.

In a further experiment, hard gelatine capsule shells (size 1) were filled with 282 mg of ammonio methacrylate copolymer Type A as a powder (Eudragit® RL PO) without any other ingredient. After storage for 3 weeks at 40° C./75% relative humidity in induction-sealed HDPE bottles, the capsule shells were opened, emptied and filled with eletriptan immediate release beads (corresponding to 40 mg eletriptan) and eletriptan controlled release beads (corresponding to 40 mg eletriptan. The capsule shells were closed again and subjected to a dissolution test. A non-uniform pattern of drug release was observed.

These experiments show that crosslinking of capsule shells does not only happen specifically with an eletriptan dual release formulation but generally with all formulations containing ammonio methacrylate copolymers, independently of the active ingredient used and even if no active is present.

### EXAMPLE 5

### Incorporation of a Separate Quenching Layer Comprising Gelatine as a Quenching Agent to Stabilise an Eletriptan Hemisuphate Dual Release Formulation

Eletriptan hemisulfate starter cores were prepared and coated with ammonio methacrylate copolymers Type A and B according to the procedures of WO-A-02/09675 using a Glatt GPCG-1 fluid-bed equipment with a 6-inch Wurster insert at a 1.5 kg batch size. Immediately after this modified-release coating step, a second barrier coat of hydroxypropylmethylcellulose was sprayed onto the beads as a 15% aqueous dispersion of Opadry II Orange 32K23388. A mixture of one part of gelatine and 10 parts of purified water was prepared at 50° C. with continuous stirring and constant temperature and sprayed onto the beads at a spray rate of 2.5-7.5 g/minute. Inlet air temperature was at about 60° C., outlet air temperature and product temperature were kept at about 50° C. Immediately after this gelatine layer coating step, a top layer of hydroxypropylmethylcellulose was sprayed onto the beads as a 15% aqueous dispersion of Opadry II Orange 32K23388. The final bead weight was 193.03 mg.

Eletriptan hemisulfate controlled release beads with quenching agent

| Component | Description | Amount |
|---|---|---|
| Eletriptan hemisulfate starter | Core composition including | 108.04 mg |
| cores | barrier coat | |
| (corresponding to 40.00 mg | | |
| eletriptan base) | | |
| Ammonio methacrylate | Controlled release coating | 24.91 mg |
| copolymer type B (Eudragit ® | | |
| RS) | | |
| Ammonio Methacrylate | | 1.31 mg |
| Copolymer Type A | | |
| (Eudragit ® RL) | | |
| Triethylcitrate NF | | 5.24 mg |
| Talc | | 13.10 mg |
| Opadry II Orange 32K23388 | 2nd Barrier Coat | 8.92 mg |
| Gelatine | Quenching Layer | 7.21 mg |
| Opadry II Orange 32K23388 | Top Coat | 22.30 mg |

The coated Modified-Release beads were cured in a tray oven at 40° C. for 24 hours and then filled together with immediate-release beads into hard gelatine capsules (size 1). The capsule fill weight was 307.99 mg.

Eletriptan hemisulfate controlled release capsule with quenching
agent

| Component | Description | Amount |
|---|---|---|
| Eletriptan hemisulfate immediate | Core composition | 116.96 mg |
| release beads (corresponding to | including top coat | |
| 40.00 mg eletriptan base) | | |
| Eletriptan hemisulfate modified | Core composition | 191.03 mg |
| release beads (corresponding to | including barrier | |
| 40.00 mg eletriptan base) | coats, quenching | |
| | layer and top coat | |
| Hard gelatine capsule shell (size 1), | Capsule shell | 76 mg |
| two-tone grey | | |

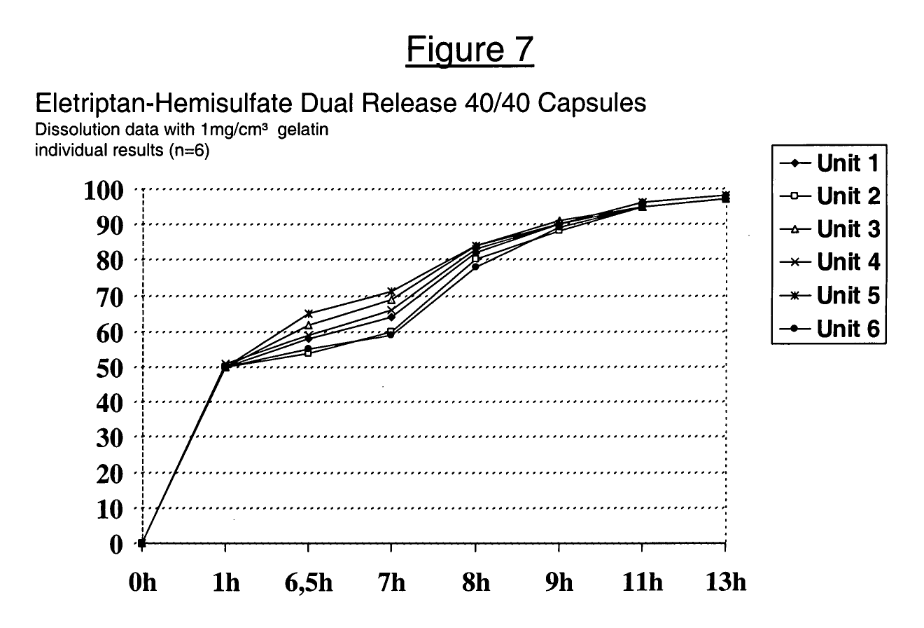

The release profile as shown in FIG. 7 was obtained using the dissolution test described in Example 6 of WO-A-02/09675.

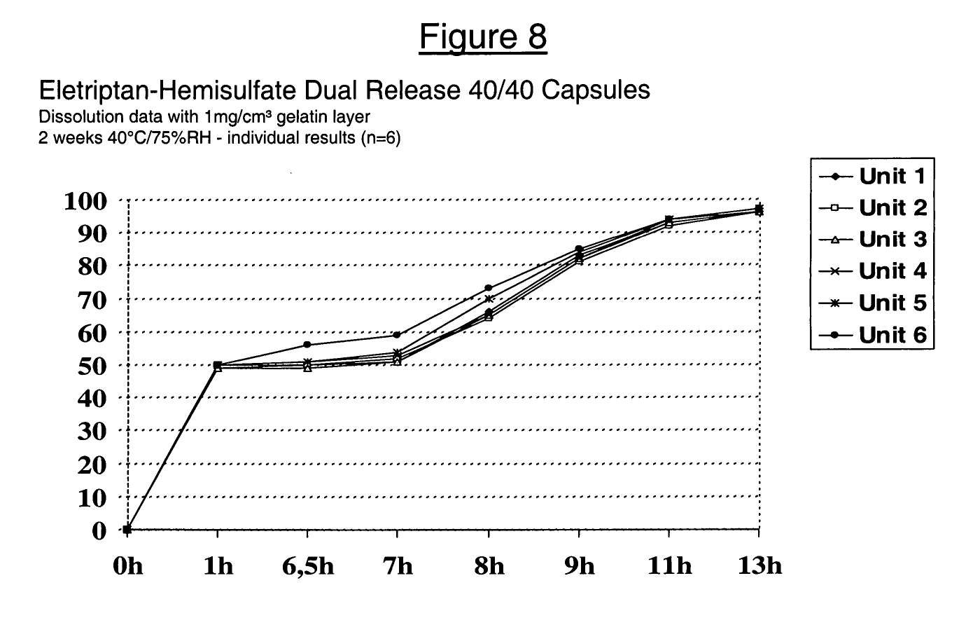

After storage for 2 weeks at 40° C./75% relative humidity in induction-sealed HDPE bottles the dissolution test was repeated and no significant change in release profile was observed. The results are shown in FIG. 8.

This experiment demonstrates that the use of a quenching layer comprising gelatine reduces any deleterious interaction between ammonio methacrylate-containing controlled release beads and the gelatine capsule and provides for a formulation which is significantly more stable upon storage.

### EXAMPLE 6

### Incorporation of Cysteine as a Quenching Agent in the Top Coat to Stabilise an Eletriptan Hemisuphate Dual Release Formulation

Eletriptan hemisulfate starter cores were prepared and coated with ammonio methacrylate copolymers type A and B according to the disclosure of WO-A-02/09675 using a Glatt GPCG-1 fluid-bed equipment with a 6-inch Wurster insert at a 1.4 kg batch size. A 0.4% solution of L-cysteine in purified water was prepared using a blade stirrer. Under continous stirring, 3 parts of Opadry II Orange powder blend were added to 17 parts of the aqueous L-cysteine solution and dispersed. The dispersion was sprayed onto the beads at a spray rate of 21 g/min. Inlet air temperature was at about 60° C., outlet air temperature and product temperature were kept at about 40° C. The final bead weight was 175.40 mg.

Eletriptan hemisulfate controlled release beads with quenching agent

| Component | Description | Amount |
|---|---|---|
| Eletriptan hemisulfate starter | Core composition including | 108.04 mg |
| cores | barrier coat | |
| (corresponding to 40.00 mg | | |
| eletriptan base) | | |
| Ammonio methacrylate | Controlled release layer | 24.91 mg |
| copolymer type B (Eudragit ® | | |
| RS) | | |
| Ammonio methacrylate | | 1.31 mg |
| copolymer type A (Eudragit ® | | |
| RL) | | |
| Triethylcitrate NF | | 5.24 mg |
| Talc | | 13.10 mg |
| Opadry II Orange 32K23388 | Top Coat including | 22.30 mg |
| L-Cysteine | Quenching Agent | 0.5 mg |

The coated controlled release beads were cured in a tray oven at 40° C. for 24 hours and filled together with immediate-release beads into hard gelatine capsules (size 1). The capsule fill weight was 292.36 mg.

Eletriptan hemisulfate dual release capsule with quenching agent

| Component | Description | Amount |
|---|---|---|
| Eletriptan hemisulfate | Core composition including | 116.96 mg |
| immediate-release beads | top coat | |
| (corresponding to 40.00 mg | | |
| eletriptan base) | | |
| Eletriptan hemisulfate | Core composition including | 175.40 mg |
| modified-release beads | barrier coat, controlled | |
| (corresponding to 40.00 mg | release layer, and top coat | |
| eletriptan base) | with quenching agent | |
| Hard gelatin capsule (shell | Capsule shell | 76 mg |
| size 1), two-tone grey | | |

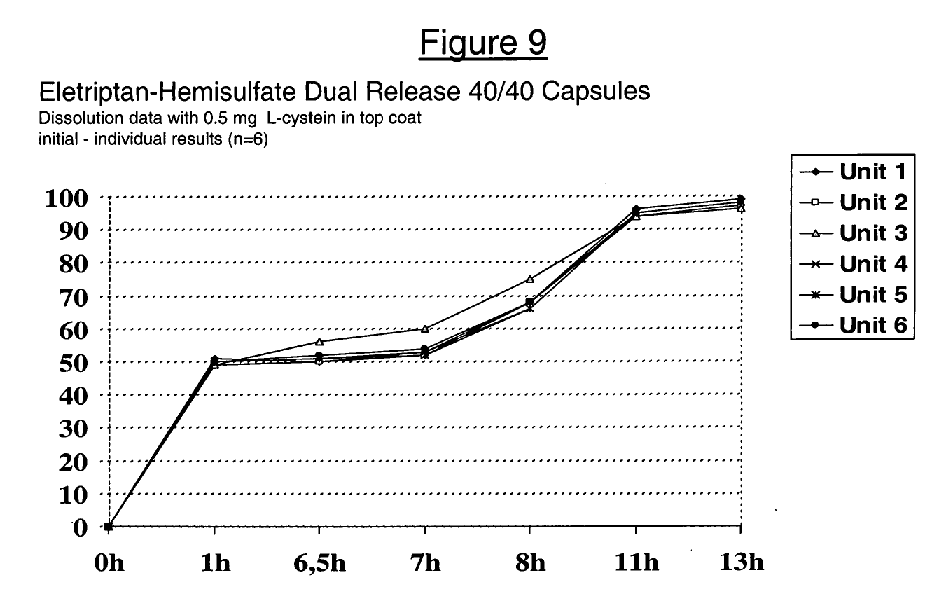

The dissolution results are shown in FIG. 9.

The dual-release capsules were stored for 4 weeks at 40° C./75% relative humidity in induction-sealed HDPE bottles and tested for dissolution.

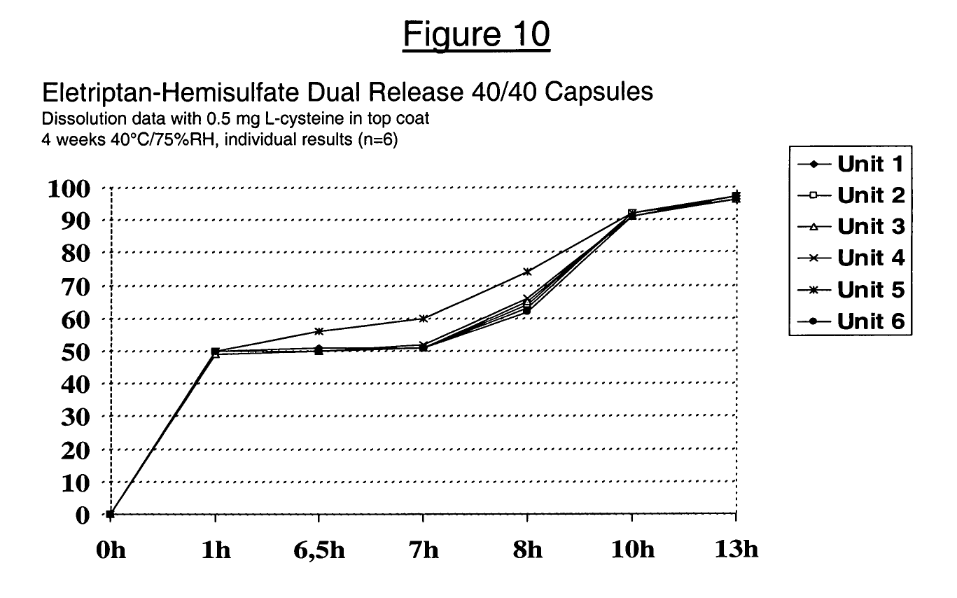

As sown in FIG. 10, this experiment demonstrates that the use of cysteine as a quenching agent in the top coat reduces any deleterious interaction between ammonio methacrylate-containing controlled release beads and the gelatine capsule and provides for a formulation which is significantly more stable upon storage.

### EXAMPLE 7

### Incorporation of Glycine as a Quenching Agent in the Top Coat to Stabilise an Eletriptan Hemisulphate Dual Release Formulation

Eletriptan hemisulfate starter cores were prepared and coated with ammonio methacrylate copolymers type A and B according to the procedures of WO-A-02/09675 using a Glatt GPCG-1 fluid-bed equipment with a 6-inch Wurster insert at a 1.6 kg batch size. An 0.4% solution of glycine in purified water was prepared using a blade stirrer. Under continous stirring, 3 parts of Opadry II Orange powder blend were added to 17 parts of the aqueous glycine solution and dispersed. The dispersion was sprayed onto the beads at a spray rate of 16-20 g/min. Inlet air temperature was at about 60° C., outlet air temperature and product temperature were kept at about 40° C. The final bead weight was 1 95.97 mg.

Eletriptan hemisulfate controlled release beads with quenching agent

| Component | Description | Amount |
|---|---|---|
| Eletriptan hemisulfate starter | Core composition including | 108.04 mg |
| cores (corresponding to 40.00 | barrier coat | |
| mg eletriptan base) | | |
| Ammonio methacrylate | Controlled release layer | 36.40 mg |
| copolymer type B (Eudragit ® | | |
| RS) | | |
| Ammonio methacrylate | | 1.92 mg |
| copolymer type A (Eudragit ® | | |
| RL) | | |
| Triethylcitrate NF | | 7.66 mg |
| Talc | | 19.15 mg |
| Opadry II Orange 32K23388 | Top coat with quenching | 22.30 mg |
| Glycine | agent | 0.5 mg |

The coated Modified-Release beads were cured in a tray oven at 40° C. for 24 hours and filled together with immediate-release beads into hard gelatine capsules (size 1). The capsule fill weight was 312.93 mg.

Eletriptan hemisulfate dual release capsule with quenching agent

| Component | Description | Amount |
|---|---|---|
| Eletriptan hemisulfate | Core composition including | 116.96 mg |
| immediate-release beads | top coat | |
| (corresponding to 40.00 mg | | |
| eletriptan base) | | |
| Eletriptan hemisulfate | Core composition including | 195.97 mg |
| modified-release beads | barrier coat, controlled | |
| (corresponding to 40.00 mg | release layer and top coat | |
| eletriptan base) | with quenching agent | |
| Hard gelatine capsule shell | Capsule shell | 76 mg |
| (size 1), two-tone grey | | |

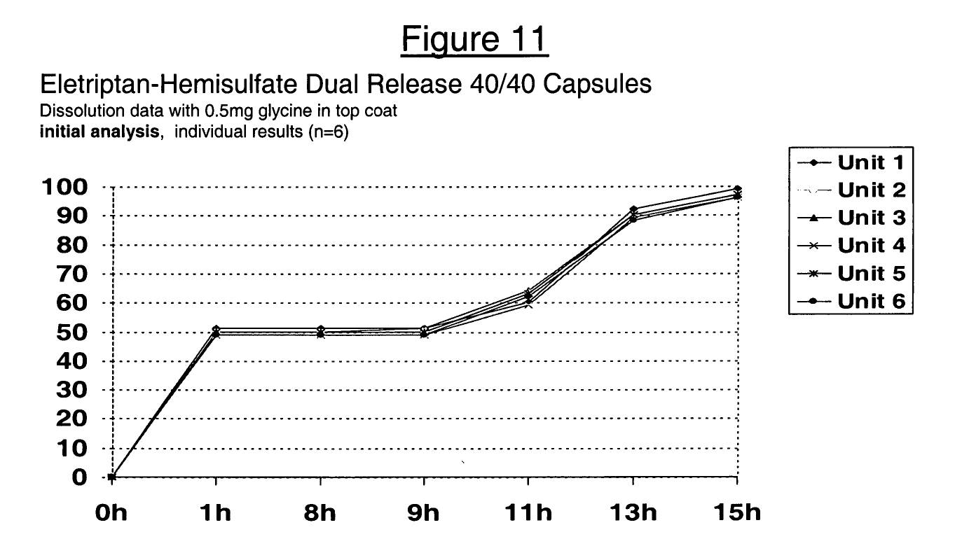

The dissolution results are shown in FIG. 11.

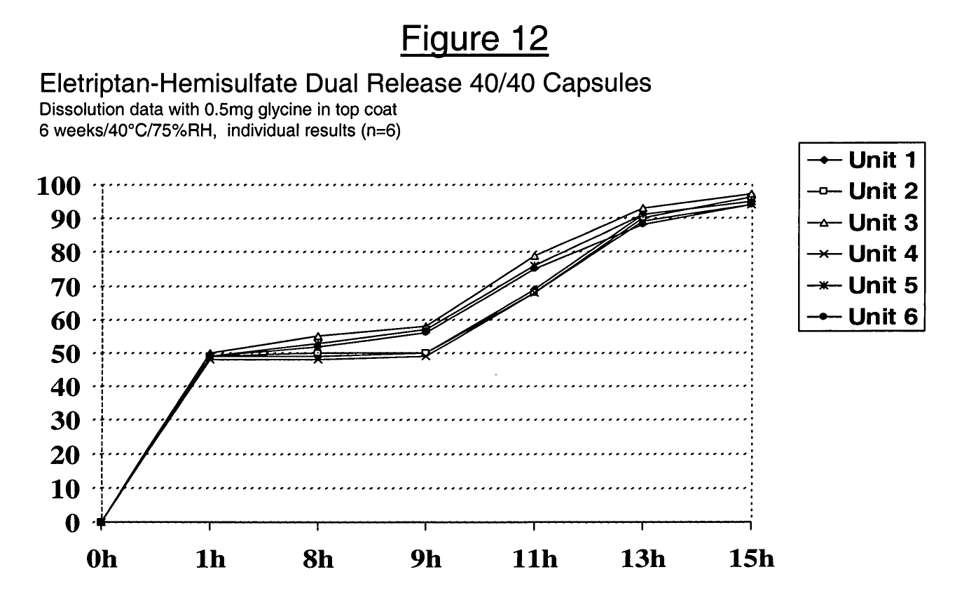

The dual release capsules were stored for 6 weeks at 40° C./75% relative humidity in induction-sealed HDPE bottles and tested for dissolution. The results are shown in FIG. 12.

This experiment demonstrates that the use of glycine as a quenching agent in the top coat reduces any deleterious interaction between ammonio methacrylate-containing controlled release beads and the gelatine capsule and provides for a formulation which is significantly more stable upon storage.

### EXAMPLE 8

### Incorporation of Glycine as a Quenching Agent in the Controlled Release Layer to Stabilise an Eletriptan Hemisulphate Dual Release Formulation

An aqueous dispersion of talc was prepared using a rotor-stator stirrer (Ultra-Turrax®). Glycine was dissolved in purified water using a blade stirrer, triethylcitrate was added and dissolved, 30% aqueous dispersions of ammonio methacrylate copolymer dispersions type A (Eudragit®RL) and B (Eudragit®RS) were added and dispersed. The aqueous talc dispersion and the aqueous ammonio methacrylate copolymer dispersion were combined and sprayed onto eletriptan hemisulfate starter cores using a Glatt GPCG-1 fluid-bed equipment with a 6-inch Wurster insert at a 1.6-kg batch size. A 15% aqueous Opadry II Orange dispersion was sprayed onto the modified-release beads. The final bead weight was 195.97 mg.

Eletriptan hemisulfate controlled release beads with quenching agent

| Component | Description | Amount |
|---|---|---|
| Eletriptan hemisulfate | Core composition | 108.04 mg |
| starter cores | including top coat | |
| (corresponding to 40.00 | | |
| mg eletriptan base) | | |
| Ammonio methacrylate | Controlled release | 36.40 mg |
| copolymer type B | layer including | |
| (Eudragit ® RS) | quenching agent | |
| Ammonio methacrylate | | 1.92 mg |
| copolymer type A | | |
| (Eudragit ® RL) | | |
| Triethylcitrate NF | | 7.66 mg |
| Glycine | | 0.50 mg |
| Talc | | 19.15 mg |
| Opadry II Orange 32K23388 | Top coat | 22.30 mg |

The coated controlled release beads were cured in a tray oven at 40° C. for 24 hours and filled together with immediate-release beads into hard gelatine capsules (size 1). The capsule fill weight was 312.93 mg.

Eletriptan hemisulfate dual release capsule with quenching agent

| Component | Description | Amount |
|---|---|---|
| Eletriptan hemisulfate | Core composition | 116.96 mg |
| immediate-release beads | including top coat | |
| (corresponding to 40.00 | | |
| mg eletriptan base) | | |
| Eletriptan hemisulfate | Core composition | 195.97 mg |
| modified-release beads | including barrier | |
| (corresponding to 40.00 | coat, controlled | |
| mg eletriptan base) | release layer with | |
| | quenching agent | |
| | and top coat | |
| Hard gelatine capsule | Capsule shell | 76 mg |
| shell (size 1), | | |
| two-tone grey | | |

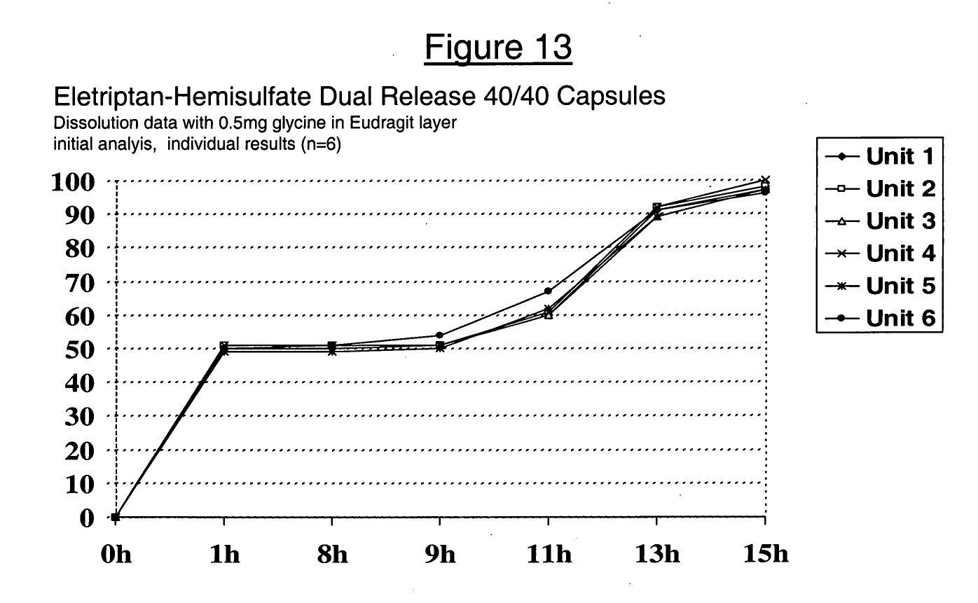

The following dissolution profile is shown in FIG. 13.

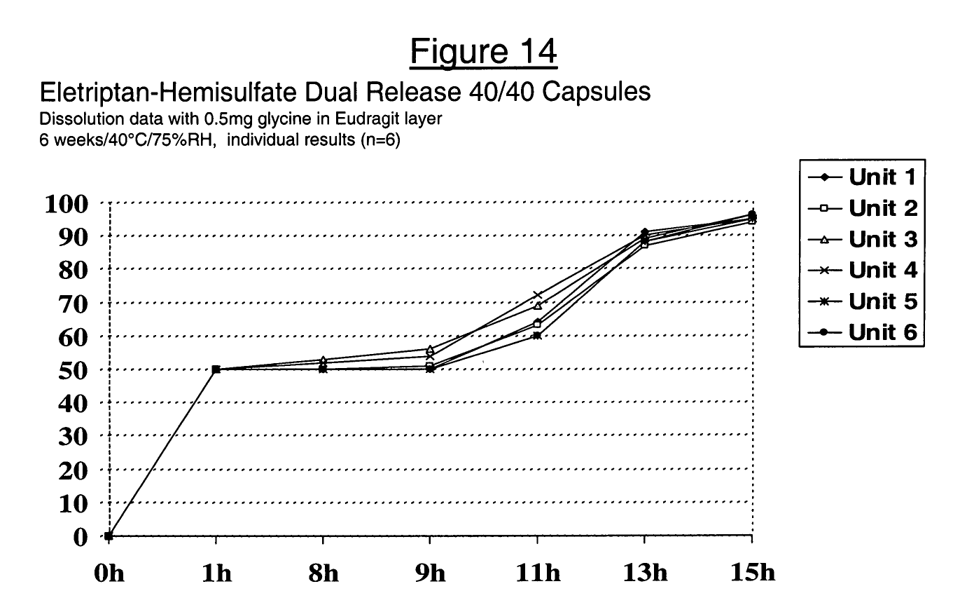

The dual-release capsules were stored for 6 weeks at 40° C./75% relative humidity in induction-sealed HDPE bottles and tested for dissolution. The results are shown in FIG. 14.

This experiment demonstrates that the use of glycine as a quenching agent in the controlled release layer reduces any deleterious interaction between ammonio methacrylate-containing controlled release beads and the gelatine capsule and provides for a formulation which is significantly more stable upon storage.

### EXAMPLE 9

### Incorporation of Asparagine as a Quenching Agent in the Top Coat to Stabilise an Eletriptan Hemisulphate Dual Release Formulation

Eletriptan hemisulfate starter cores were prepared and coated with ammonio methacrylate copolymers type A and B according to the procedures of WO-A-02/09675 using a Glatt GPCG-1 fluid-bed equipment with a 6-inch Wurster insert at a 1.6 kg batch size. A 0.4% solution of L-asparagine monohydrate in purified water was prepared using a blade stirrer. Under continous stirring, 3 parts of Opadry II Orange powder blend were added to 17 parts of the aqueous asparagine solution and dispersed. The dispersion was sprayed onto the beads at a spray rate of about 20 g/min. Inlet air temperature was at about 60° C., outlet air temperature and product temperature were kept at about 40° C. The final bead weight was 195.97 mg.

Eletriptan hemisulfate controlled release beads
with quenching agent

| Component | Description | Amount |
|---|---|---|
| Eletriptan hemisulfate starter | Core composition including | 108.04 mg |
| cores (corresponding to 40.00 | barrier coat | |
| mg eletriptan base) | | |
| Ammonio methacrylate | Controlled release layer | 36.40 mg |
| copolymer type B (Eudragit ® | | |
| RS) | | |
| Ammonio methacrylate | | 1.92 mg |
| copolymer type A (Eudragit ® | | |
| RL) | | |
| Triethylcitrate NF | | 7.66 mg |
| Talc | | 19.15 mg |
| Opadry II Orange 32K23388 | Top coat including | 22.30 mg |
| L-Asparagine monohydrate | quenching agent | 0.5 mg |

The coated controlled release beads were cured in a tray oven at 40° C. for 24 hours and filled together with immediate-release beads into hard gelatine capsules (size 1). The capsule fill weight was 312.93 mg.

Eletriptan hemisulfate dual release capsule with quenching agent

| Component | Description | Amount |
|---|---|---|
| Eletriptan hemisulfate | Core composition including | 116.96 mg |
| immediate-release beads | top coat | |
| (corresponding to 40.00 mg | | |
| eletriptan base) | | |
| Eletriptan hemisulfate | Core composition including | 195.97 mg |
| modified-release beads | barrier coat, controlled | |
| (corresponding to 40.00 mg | release layer and top coat | |
| eletriptan base) | with quenching agent | |
| Hard gelatine capsule shell | Capsule shell | 76 mg |
| (size 1), two-tone grey | | |

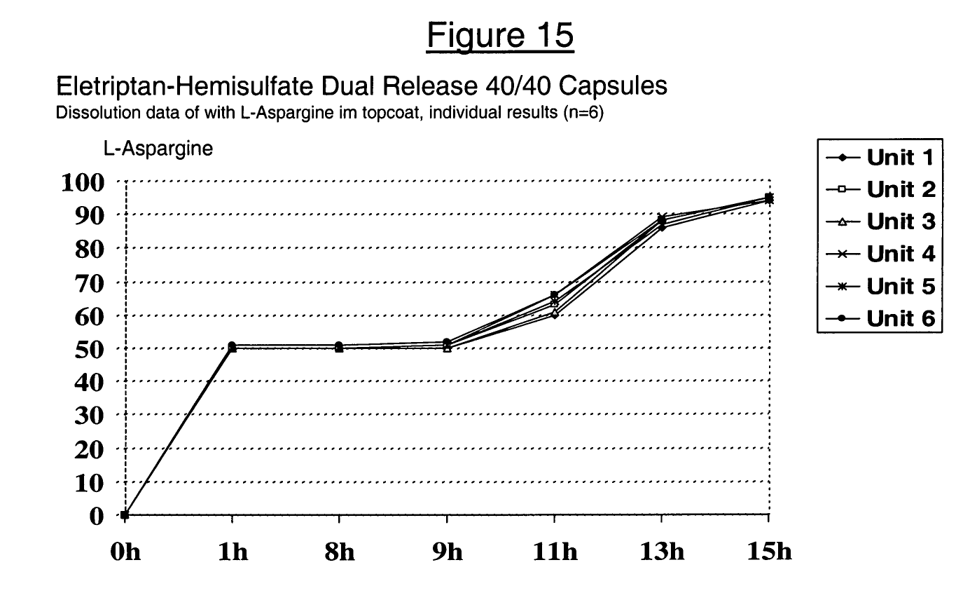

The dissolution results are shown in FIG. 15.

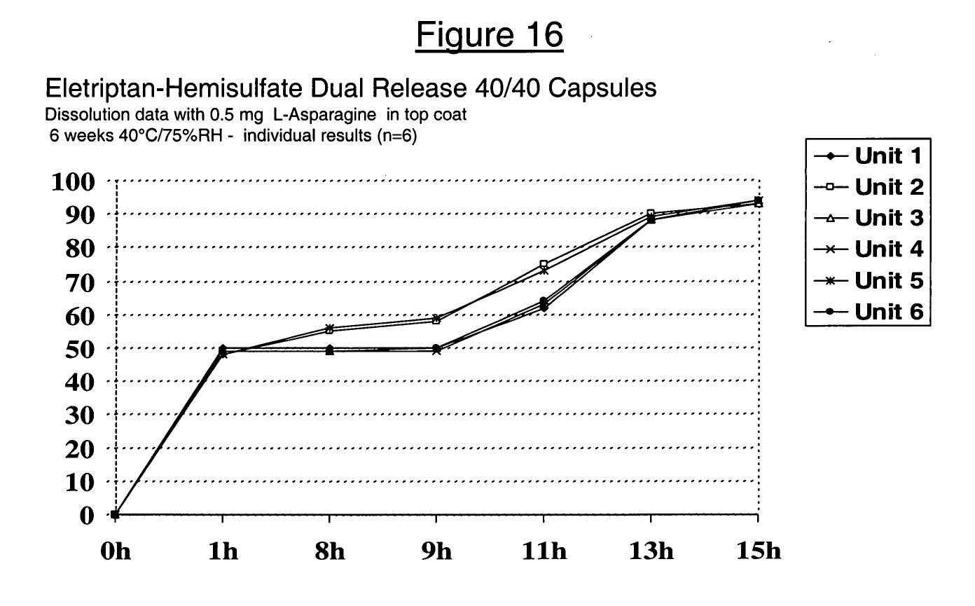

The dual release capsules were stored for 6 weeks at 40° C./75% relative humidity in induction-sealed HDPE bottles and tested for dissolution. The results are shown in FIG. 16.

This experiment demonstrates that the use of asparagine as a quenching agent in the top coat reduces any deleterious interaction between ammonio methacrylate-containing controlled release beads and the gelatine capsule and provides for a formulation- which is significantly more stable upon storage.

### EXAMPLE 10

### Incorporation of Asparagine as a Quenching Agent in the Controlled Release Layer to Stabilise an Eletriptan Hemisulphate Dual Release Formulation

An aqueous dispersion of talc was prepared using a rotor-stator stirrer (Ultra-Turrax®). L-asparagine monohydrate was dissolved in purified water using a blade stirrer, triethylcitrate was added and dissolved, 30% aqueous dispersions of ammonio methacrylate copolymer dispersions type A (Eudragit® RL) and B (Eudragit® RS) were added and dispersed. The aqueous talc dispersion and the aqueous ammonio methacrylate copolymer dispersion were combined and sprayed onto eletriptan hemisulfate starter cores using a Glatt GPCG-1 fluid-bed equipment with a 6-inch Wurster insert at a 1.6-kg batch size. A 15% aqueous Opadry II Orange dispersion was sprayed onto the controlled release beads. The final bead weight was 197.97 mg.

Eletriptan hemisulfate controlled release beads
with quenching agent

| Component | Description | Amount |
|---|---|---|
| Eletriptan hemisulfate starter | Core composition including | 108.04 mg |
| cores (corresponding to 40.00 | barrier coat | |
| mg eletriptan base) | | |
| Ammonio methacrylate | Controlled release layer | 36.40 mg |
| copolymer type B (Eudragit ® | including quenching agent | |
| RS) | | |
| Ammonio methacrylate | | 1.92 mg |
| copolymer type A (Eudragit ® | | |
| RL) | | |
| Triethylcitrate NF | | 7.66 mg |
| L-Asparagine monohydrate | | 2.50 mg |
| Talc | | 19.15 mg |
| Opadry II Orange 32K23388 | Top coat | 22.30 mg |

The coated modified-release beads were cured in a tray oven at 40° C. for 24 hours and filled together with immediate-release beads into hard gelatine capsules (size 1). The capsule fill weight was 314.93 mg.

Eletriptan hemisulfate dual release capsule

| Component | Description | Amount |
|---|---|---|
| Eletriptan hemisulfate | Core composition including | 116.96 mg |
| immediate-release beads | top coat | |
| (corresponding to 40.00 mg | | |
| eletriptan base) | | |
| Eletriptan hemisulfate | Core composition including | 197.97 mg |
| controlled release beads | barrier coat, controlled | |
| (corresponding to 40.00 mg | release layer with quenching | |
| eletriptan base) | agent and top coat | |
| Hard gelatine capsule shell | Capsule shell | 76 mg |
| (size 1), two-tone grey | | |

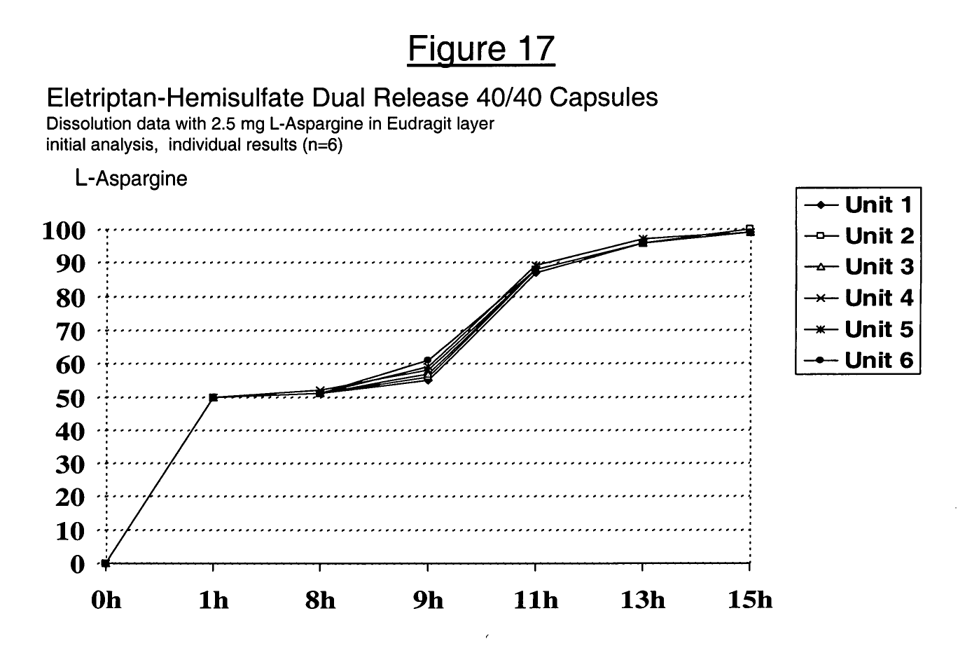

The dissolution results are shown in FIG. 17.

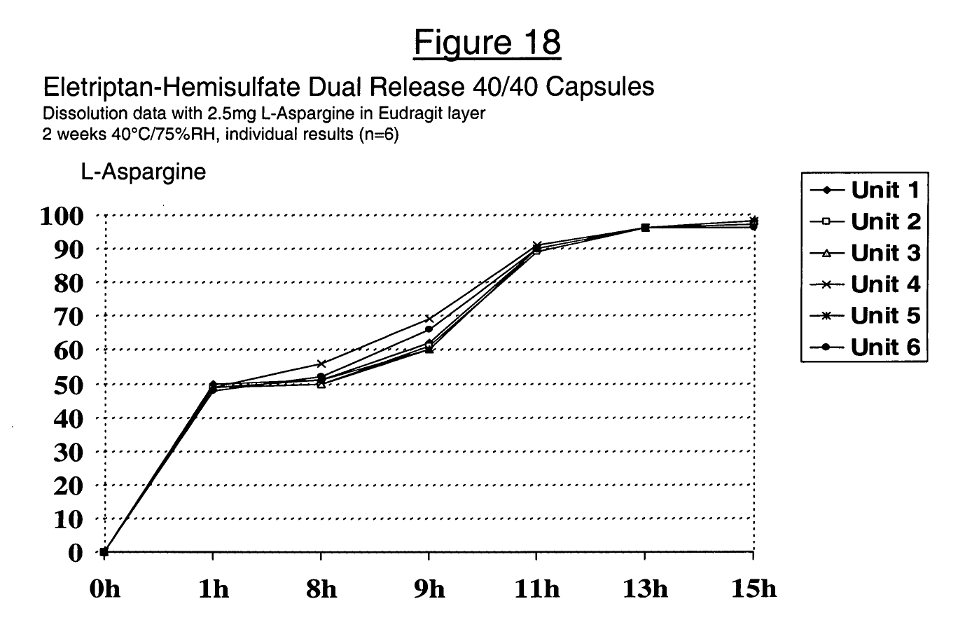

The dual-release capsules were stored for 2 weeks at 40° C./75% relative humidity in induction-sealed HDPE bottles and tested for dissolution. The results are shown in FIG. 18.

This experiment demonstrates that the use of asparagine as a quenching agent in the controlled release layer reduces any deleterious interaction between ammonio methacrylate-containing controlled release beads and the gelatine capsule and provides for a formulation which is significantly more stable upon storage.

1. A pharmaceutical composition in gelatine capsule form, comprising (a) an active core composition comprising at least one pharmaceutically active compound and optionally conventional pharmaceutical adjuvants, (b) at least one quenching protective layer, positioned between said active core composition and the inner surface of the gelatine capsule shell, which comprises at least one quenching agent, the quenching agent being able to chemically bind volatile impurities initiating the cross-linking of gelatine by a substantially irreversible chemical reaction after said impurities have been released by either adjuvants and/or active compound(s) of said active core composition and (c) a gelatine capsule shell wrapping said active core composition including any adjuvant material and any quenching layer added to the active core composition.

2. A pharmaceutical composition as claimed in claim 1 wherein the active core composition comprises an ammonio methacrylate copolymer such as Eudragit® RL 30 D (type A) or Eudragit® RS 30 D (type B).

3. A pharmaceutical composition as claimed in claim 2 wherein the ammonio methacrylate copolymer is present as a controlled release coating on the external surface of the active core composition.

4. A pharmaceutical composition as claimed in claim 1 wherein the quenching agent is gelatine, and/or an amino-acid, or combination thereof.

5. A pharmaceutical composition as claimed in claim 4 wherein the quenching agent is cysteine, lysine, aspartic acid, asparagine, gelatine or glycine.

6. A pharmaceutical composition as claimed in claim 1, wherein the quenching protective layer, comprising the quenching agent, is positioned next to or coated onto the inner surface of the gelatine capsule shell.

7. A pharmaceutical composition as claimed in claim 1, wherein the quenching protective layer, comprising the quenching agent, is positioned next to or coated onto the outer surface of the active core composition.

8. A pharmaceutical composition as claimed in claim 3, wherein the quenching agent is integrated into the controlled release layer to form a bifunctional layer.

9. A pharmaceutical formulation as claimed in claim 1, wherein the capsule contains a dual release formulation comprising immediate release particles and controlled release particles.

10. A pharmaceutical composition as claimed in claim 1, wherein the active core composition comprises eletriptan, or a pharmaceutically acceptable salt or solvate thereof.

11. A pharmaceutical composition as claimed in claim 10, wherein the active core composition comprises eletriptan hemisulphate.

12. A method of treating migraine or preventing migraine recurrence in a mammal, including a human, said method comprising administration to said mammal of a therapeutically effective amount of a controlled release pharmaceutical formulation as claimed in claim 10.

13. A multiparticulate composition, suitable for administration in a gelatine capsule, comprising a drug-containing core, said core being coated with a controlled release coating comprising an ammonio methacrylate copolymer, wherein the composition comprises a quenching agent in the controlled release coating or in an outer quenching protective layer.

14. A method for improving the stability of a pharmaceutical gelatine-capsule preparation by preventing or reducing the cross-linking of the gelatine material in the capsule shell initiated by volatile impurities that are released by (a) preparing a quenching protective layer comprising at least one quenching-agent which is able to chemically bind volatile impurities initiating the cross-linking of gelatine, by a substantially irreversible chemical reaction and (b) positioning said quenching layer in the space between the pharmaceutical core compositions or its components releasing volatile impurities and the inner surface of the gelatine-capsule shell in a way that said volatile impurities are forced to contact and penetrate the surface of said quenching protective layer before said volatile impurities are able to contact the inner surface of the gelatine capsule shell.
